(19) United States
(12) Patent Application Publication
(10) Pub. No.: US 2004/0110845 A1
(43) Pub. Date: Jun. 10, 2004

(54) Stabilized albuterol compositions and method of preparation thereof

(76) Inventors: Ramana Malladi, Marietta, GA (US); Partha S. Banerjee, San Ramon, CA (US); Satyajit V. Erram, Fairfield, CA (US)

Correspondence Address: HELLER EHRMAN WHITE & MCAULIFFE LLP, 4350 LA JOLLA VILLAGE DRIVE, 7TH FLOOR, SAN DIEGO, CA, 92122-1246, US

(21) Appl. No.: 10/314,107

(22) Filed: Dec. 6, 2002

(51) Int. Cl. A61K 31/137

(52) U.S. Cl. 514/651

(57) ABSTRACT

Stabilized albuterol compositions are provided. The compositions are aqueous inhalation compositions containing albuterol; a buffer, such as citric acid; and a metal chelator, such as EDTA.

### FIELD

Compositions are provided containing albuterol having a long shelf life with reduced levels of albuterol aldehyde degradation product. Methods of preparing the compositions are also provided.

### BACKGROUND

Albuterol is a β-adrenergic agonist and is indicated for the treatment of bronchoconstrictive disorders. Albuterol, also referred to as salbutamol, has the formula:

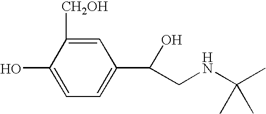

and is described in U.S. Pat. No. 3,705,233.

Albuterol undergoes degradation in aqueous aolutions to albuterol aldehyde. Albuterol aldehyde has potential negative effects when administered by inhalation and therefore its level in inhalation solutions is controlled by the U.S. Food and Drug Administration. The rate of degradation of albuterol in aqueous solutions, to albuterol aldehyde, increases with increasing initial drug concentation (Malkki et al. (1990) Int. J. Pharmaceutics 63:17-22).

Antioxidants such as thiourea reduce the oxidative decomposition of albuterol in aqueous solutions to albuterol aldehyde (Malkki et al., supra). However, addition of sulfur containing antioxidants such as thiourea is not recommended for formulations intended for inhalation.

One approach to reduce the levels of albuterol aldehyde described earlier has been to blow nitrogen gas over the solution during formulation and filling of the solution in unit dose vials. The vials are then enclosed in an oxygen-impermeable wrapper in a reduce oxygen atmosphere (U.S. Pat. No. 6,451,289). This process is cumbersome and it is difficult to control the level of oxygen during manufacturing and storage. Once the protective wrapper is opened, albuterol is exposed to ambient oxygen and degradation can occur.

Therefore, there is a need to provide stabilized albuterol compositions. There is also a need to provide methods of stabilizing albuterol compositions.

### SUMMARY

Provided herein are stabilized albuterol compositions. Methods of stabilizing albuterol compositions are also provided. In another embodiment, methods of preparation of stabilized albuterol compositions are provided. The stabilized compositions have a long shelf life.

In one embodiment, the compositions are pharmaceutical compositions containing albuterol, or a derivative thereof; a buffer; and a metal chelator. In another embodiment, the compositions are aqueous compositions. In another embodiment, the compositions are aqueous inhalation solutions. The compositions possess a long shelf life. Thus, the compositions maintain their stability for at least 6 months at 40° C., or at least 18 months at 25° C. In these embodiments, the compositions contain less that 0.01% by weight of albuterol aldehyde after storage for 6 months at 40° C., or storage for 18 months at 25° C. In certain embodiments herein, the compositions are formulated under ambient atmosphere (i.e., without nitrogen gas sparging of the formulation or replacement of the headspace atmosphere with nitrogen gas in the storage container).

Buffers for use herein include citric acid, citrate buffer, citric acid/phosphate buffer, phosphate-acetate-borate buffer (Britton-Robinson), and citrate-phosphate-borate buffer (Teorell-Stanhagen).

Metal chelators for use herein include, but are not limited to, ethylenediamine tetraacetic acid (EDTA, (HOOCCH₂)₂NCH₂CH₂N—(CH₂COOH)₂), nitrilotriacetic acid (N(CH₂COOH)₃), ethylene glycol-bis(β-aminoethyl ether)-N,N-tetraacetic acid (HOOCCH₂)₂NCH₂CH₂)CH₂CH₂O—CH₂CH₂N(CH₂COOH)₂), ethylene glycol bis(β-aminoethyl ether)-N,N,N′,N′-tetaacetic acid (EGTA), glycine, salicylaldehyde, albumin, pilocarpine, chlorophyll, hemoglobin, peroxidases, cytochromes, oxidases, ascorbic acid oxidase, tyrosinase, polyphenoloxidase, lactase, phosphatase, carboxylases, insulin, cyanocobalbumin, carbonic anhydrase, xanthine dehydrogenase and tetracyclines. Metal chelators may also be employed as their pharmaceutically acceptable derivatives, including pharmaceutically acceptable salts, including sodium salts. In one embodiment, the metal chelator for use in the compositions is a pharmaceutically acceptable derivative of EDTA.

Derivatives of albuterol for use in the compositions and methods provided herein include salts, esters, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable solvates and hydrates are complexes of a compound with one or more solvent or water molecules, or 1 to about 100, or 1 to about 10, or one to about 2, 3 or 4, solvent or water molecules. In one embodiment, the pharmaceutically acceptable derivative of albuterol is a salt. In another embodiment, the pharmaceutically acceptable derivative of albuterol is a mineral acid salt. In another embodiment, the pharmaceutically acceptable derivative of albuterol is the sulfate salt, including albuterol hemisulfate, having the formula:

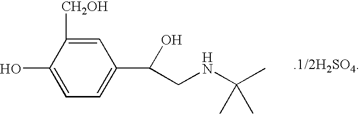

### DETAILED DESCRIPTION OF PREFERRED EMBODIMENTS

A. Definitions

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

As used herein, albuterol refers to a compound having the formula:

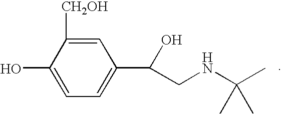

It is to be understood that albuterol contains chiral centers. Such chiral centers may be of either the (R) or (S) configuration, or may be a mixture thereof. Thus, as used herein, “albuterol” may be enantiomerically pure, or be a stereoisomeric mixture.

As used herein, the term “albuterol” includes the free base and pharmaceutically acceptable derivatives thereof, including salts thereof, including mineral acid salts thereof, including sulfate salts thereof, including the hemisulfate thereof.

As used herein, “albuterol aldehyde” refers to 5-(2-((1,1-dimethylethyl)-amino)-1-hydroxyethyl)-2-hydroxybenzaldehyde, pharmaceutically acceptable derivatives thereof, and stereoisomers thereof.

As used herein, a metal chelator is a chemical compound that forms a coordination compound (a chelate) in which a central metal ion is attached by coordinate links to two or more nonmetal atom in the same molecule. Metal chelators for use in the compositions provided herein include, but are not limited to, ethylenediamine tetraacetic acid (EDTA, (HOOCCH₂)₂NCH₂CH₂N—(CH₂COOH)₂), nitrilotriacetic acid (N(CH₂COOH)₃), ethylene glycol-bis(β-aminoethyl ether)-N,N-tetraacetic acid (HOOCCH₂)₂N CH₂CH₂)CH₂CH₂O—CH₂CH₂N(CH₂COOH)₂), ethylene glycol bis(β-aminoethyl ether)-N,N,N′,N′-tetaacetic acid (EGTA), glycine, salicylaldehyde, albumin, pilocarpine, chlorophyll, hemoglobin, peroxidases, cytochromes, oxidases, ascorbic acid oxidase, tyrosinase, polyphenoloxidase, lactase, phosphatase, carboxylases, insulin, cyanocobalbumin, carbonic anhydrase, xanthine dehydrogenase and tetracyclines. Metal chelators may also be employed as their pharmaceutically acceptable derivatives, including pharmaceutically acceptable salts, including sodium salts.

As used herein, pharmaceutically acceptable derivatives of a compound include salts, esters, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a disease or disorder are ameliorated or otherwise beneficially altered. Treatment also encompasses any pharmaceutical use of the compositions herein, such as use for treating bronchoconstrictive diseases or disorders, or diseases or disorders in which β-adrenergic activity is implicated.

As used herein, amelioration of the symptoms of a particular disorder by administration of a particular compound or pharmaceutical composition refers to any lessening, whether permanent or temporary, lasting or transient that can be attributed to or associated with administration of the composition.

As used herein, a prodrug is a compound that, upon in vivo administration, is metabolized by one or more steps or processes or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

As used herein, alkyl, alkenyl and alkynyl carbon chains, if not specified, contain from 1 to 20 carbons, or 1 or 2 to 16 carbons, and are straight or branched. Alkenyl carbon chains of from 2 to 20 carbons, in certain embodiments, contain 1 to 8 double bonds and alkenyl carbon chains of 2 to 16 carbons, in certain embodiments, contain 1 to 5 double bonds. Alkynyl carbon chains of from 2 to 20 carbons, in certain embodiments, contain 1 to 8 triple bonds, and the alkynyl carbon chains of 2 to 16 carbons, in certain embodiments, contain 1 to 5 triple bonds. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl, isohexyl, allyl (propenyl) and propargyl (propynyl). As used herein, lower alkyl, lower alkenyl, and lower alkynyl refer to carbon chains having from about 1 or about 2 carbons up to about 6 carbons.

As used herein, “cycloalkyl” refers to a saturated mono- or multi-cyclic ring system, in certain embodiments of 3 to 10 carbon atoms, in other embodiments of 3 to 6 carbon atoms. The ring systems of the cycloalkyl, groups may be composed of one ring or two or more rings which may be joined together in a fused, bridged or spiro-connected fashion.

As used herein, “aryl” refers to aromatic monocyclic or multicyclic groups containing from 6 to 19 carbon atoms. Aryl groups include, but are not limited to groups such as unsubstituted or substituted fluorenyl, unsubstituted or substituted phenyl, and unsubstituted or substituted naphthyl.

As used herein, “heteroaryl” refers to a monocyclic or multicyclic aromatic ring system, in certain embodiments, of about 5 to about 15 members where one or more, in one embodiment 1 to 3, of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, quinolinyl and isoquinolinyl.

As used herein, “heterocyclyl” refers to a monocyclic or multicyclic nonaromatic ring system, in one embodiment of 3 to 10 members, in another embodiment of 4 to 7 members, in a further embodiment of 5 to 6 members, where one or more, in certain embodiments, 1 to 3, of the atoms in the ring system is a heteroatom, that is, an element other than carbon, including but not limited to, nitrogen, oxygen or sulfur. In embodiments where the heteroatom(s) is(are) nitrogen, the nitrogen is optionally substituted with alkyl, alkenyl, alkynyl, aryl, heteroaryl, aralkyl, heteroaralkyl, cycloalkyl, heterocyclyl, cycloalkylalkyl, heterocyclylalkyl, acyl, guanidino, or the nitrogen may be quaternized to form an ammonium group where the substituents are selected as above.

As used herein, “aralkyl” refers to an alkyl group in which one of the hydrogen atoms of the alkyl is replaced by an aryl group.

As used herein, “heteroaralkyl” refers to an alkyl group in which one of the hydrogen atoms of the alkyl is replaced by a heteroaryl group.

As used herein, the abbreviations for any groups and other compounds, are, unless indicated otherwise, in accord with their common usage, recognized abbreviations, or the IUPAC-IUB Commission on Biochemical Nomenclature (see, (1972) Biochem. 11:942-944).

B. Compositions

Stabilized albuterol compositions are provided herein. The compositions contain albuterol, or a pharmaceutically acceptable derivative thereof; a buffer; and a metal chelator. Other ingredients may be added as described elsewhere herein. In one embodiment, the compositions contain a pharmaceutically acceptable salt of albuterol. In another embodiment, the compositions contain a pharmaceutically acceptable mineral acid salt of albuterol. In another embodiment, the compositions contain a sulfate salt of albuterol, including the hemisulfate having the formula:

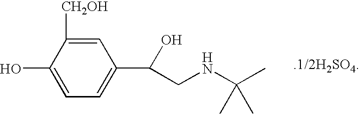

In one embodiment, the compositions contain a buffer. Buffers for use in the compositions include, but are not limited to, citric acid and citrate buffer.

In one embodiment, the compositions contain a metal chelator. Metal chelators for use in the compositions provided herein include, but are not limited to, pharmaceutically acceptable derivatives of EDTA. In another embodiment, the metal chelator is a pharmaceutically acceptable salt of EDTA. In another embodiment, the metal chelator is a sodium salt of EDTA, including the disodium salt of EDTA.

In one embodiment, the compositions contain from 0.01 to 0.75 weight % albuterol (measured as the free base). In another embodiment, the compositions contain from 0.01 to 0.5 weight % albuterol (measured as the free base). In another embodiment, the compositions contain 0.5 weight % albuterol (measured as the free base). In another embodiment, the compositions contain 0.6 weight % albuterol (measured as the hemisulfate).

In another embodiment, the compositions contain from 0.001 to 0.25 weight % EDTA (measured as the disodium salt). In another embodiment, the compositions contain from 0.005 to 0.25 weight % EDTA (measured as the disodium salt). In another embodiment, the compositions contain 0.01 weight % EDTA (measured as the disodium salt). In another embodiment, the compositions contain 0.005 weight % EDTA (measured as the disodium salt).

In another embodiment, the compositions contain from 0.005 to 0.25 weight % citric acid (measured as the free acid). In another embodiment, the compositions contain 0.01 weight % citric acid (measured as the free acid).

In other embodiments, the compositions provided herein are aqueous compositions for inhalation. The compositions, in certain embodiments herein, have a pH of from about 3.0 up to about 5.0. In another embodiment, the compositions have a pH of from about 3.0 up to about 4.0. In another embodiment, the compositions have a pH of from about 3.5 up to about 3.6. In another embodiment, the compositions have a pH of about 3.5.

In another embodiment, the compositions provided herein are aqueous inhalation compositions containing albuterol hemisulfate; citric acid; and EDTA disodium salt. It has been found herein that albuterol compositions containing citric acid and EDTA are more stable that albuterol compositions containing only citric acid or only EDTA. See, e.g., the EXAMPLES.

C. Preparation of the Compositions

The compositions are prepared by mixing of the desired ingredients in the desired amounts in a stirred vessel. The synthesis of albuterol is disclosed in U.S. Pat. No. 3,705,233, the disclosure of which is incorporated by reference herein in its entirety. In one embodiment, the compositions are prepared in a glass or stainless steel vessel. The compositions may be filled into unit dose low density polyethylene (LDPE) or polypropylene (PP) vials by form-fill-seal (FFS) technology. The unit dose vials are then packaged in a foil laminate pouch.

In one embodiment, the pharmaceutical compositions provided herein are aqueous compositions. In this embodiment, the composition is prepared by (i) dissolving the metal chelator in water; (ii) adding buffer to the metal chelator solution; and (iii) adding albuterol to the buffer/metal chelator solution.

D. Formulation of Pharmaceutical Compositions

In one embodiment, the pharmaceutical compositions provided herein contain therapeutically effective amounts of albuterol, or a pharmaceutically acceptable derivative thereof, that is useful in the prevention, treatment, or amelioration of one or more of the symptoms of diseases or disorders associated with β-adrenergic activity, or in which β-adrenergic activity is implicated; a buffer; and a metal chelator; in a pharmaceutically acceptable carrier. Diseases or disorders associated with β-adrenergic activity include, but are not limited to, bronchoconstrictive disorders, including asthma (including bronchial asthma, allergic asthma and intrinsic asthma, e.g., late asthma and airway hyper-responsiveness), chronic bronchitis and other chronic obstructive pulmonary diseases. Pharmaceutical carriers suitable for administration of the compounds provided herein include any such carriers known to those skilled in the art to be suitable for the particular mode of administration. The compositions provided herein are intended for administration via inhalation. In one embodiment, the compositions are intended for administration via nebulization.

In addition, the compounds may be formulated as the sole pharmaceutically active ingredient in the composition or may be combined with other active ingredients.

In one embodiment, an effective concentration of albuterol; a buffer; and a metal chelator; are mixed with a suitable pharmaceutical carrier. Albuterol may be derivatized as the corresponding salts, esters, solvates, hydrates or prodrugs prior to formulation, as described above. The concentrations of albuterol in the compositions are effective for delivery of an amount, upon administration, that treats, prevents, or ameliorates one or more of the symptoms of diseases or disorders associated with β-adrenergic activity or in which β-adrenergic activity is implicated. For example, in one embodiment, the compositions are single dosage compositions that contain about 0.5 mg up to about 8.0 mg of albuterol. In another embodiment, the compositions are single dosage compositions that contain about 0.63 mg, 1.25 mg or 2.5 mg of albuterol. The single dosage compositions may be administered up to 3 or 4 times per day, resulting in a total daily dosage of about 1.89 mg up to about 32 mg. In certain embodiment, the total daily dosage of albuterol is abouo 10 mg per day. For pediatric indications, the compositions contain, in one embodiment, about 0.63 mg or about 1.25 mg of albuterol, and are administered 3 or 4 times per day, resulting in a total daily dosage of about 1.89 mg up to about 5 mg of albuterol.

In one embodiment, the compositions are formulated for single dosage administration. To formulate a composition, the weight fraction of compound is dissolved, suspended, dispersed or otherwise mixed in a selected carrier at an effective concentration such that the treated condition is relieved, prevented, or one or more symptoms are ameliorated.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems described herein and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of diseases or disorders associated with β-adrenergic activity or in which β-adrenergic activity is implicated, as described herein.

Actual methods of preparing such dosage forms are known, or will be apparent, to those skilled in this art; for example, see Remington's Pharmaceutical Sciences, Mack Publishing Company, Easton, Pa., 15th Edition, 1975.

E. Combinations and Kits

Combinations and kits containing the compositions, packaged into suitable packaging material are provided. A kit typically includes a label or packaging insert including a description of the components or instructions for use (e.g., for treatment of a bronchconstrictive disorder) of the components therein. A kit can contain a collection of such components.

Kits therefore optionally include labels or instructions for using the kit components in a method provided herein. Instructions can include instructions for practicing any of the methods.

The instructions can be on “printed matter,” e.g., on paper or cardboard within the kit, or on a label affixed to the kit or packaging material, or attached to a vial or tube containing a component of the kit. Instructions can additionally be included on a computer readable medium, such as a disk (floppy diskette or hard disk), optical CD such as CD- or DVD-ROM/RAM, magnetic tape, electrical storage media such as RAM and ROM and hybrids of these such as magnetic/optical storage media. Kits can additionally include buffering agent, a preservative, or a stabilizing agent. Each component of the kit can be enclosed within an individual container and all of the various containers can be within a single package. Kits can be designed for cold storage.

In one embodiment, the kits provided herein contain an albuterol composition provided herein and a nebulizer.

F. Evaluation of the Activity of the Compositions

Standard physiological, pharmacological and biochemical procedures are available for testing the compositions provided herein to identify those that possess bronchdilatory activity.

In vitro and in vivo assays that may be used to evaluate bronchodilatory activity are well known to those of skill in the art. See also, e.g., U.S. Pat. Nos. 3,994,974, and 6,068,833; German Patent No. 2,305,092; Kaumann et al. (1985) Naunyn-Schmied Arch. Pharmacol. 331:27-39; Lemoine et al. (1985) Naunyn-Schmied Arch. Pharmacol. 331:40-51; Tomioka et al. (1981) Arch. Int. Pharmacodyn. 250:279-292; Dellamary et al. (2000) Pharm. Res. 17(2):168-174; Rico-Mendez et al. (1999) Rev. Alerg. Mex. 46(5):130-135; Seberova et al. (2000) Respir. Med. 94(6):607-611; Lotvall et al. (1999) Can. Respir. J. 6(5):412-416; Campbell et al. (1999) Respir. Med. 93(4):236-244; Nightingale et al. (1999) Am. J. Respir. Crit. Care Med. 159(6):1786-1790; Lecaillon et al. (1999) Eur. J. Clin. Pharmacol. 55(2):131-138; Bartow et al. (1998) Drugs 55(2):303-322; Ekstrom et al. (1998) Respir. Med. 92(8):1040-1045; Ringdal et a. (1998) Respir. Med. 92(8):1017-1021; Totterman et al., (1998) Eur. Respir. J. 12(3):573-579; Palmqvist et al. (1997) Eur. Respir. J. 10(11):2484-2489; Nielsen et a. (1997) Eur. Respir. J. 10(9):2105-2109; Ullman et a. (1996) Allergy 51(10):745-748; Selroos et al. (1996) Clin. Immunother. 6:273-299; and Schreurs et al. (1996) Eur. Respir. J. 9(8):1678-1683.

G. Methods of Treatment of Bronchoconstrictive Disorders

The compositions provided herein are used for treating, preventing, or ameliorating one or more symptoms of a bronchoconstrictive disorders in a mammal. In one embodiment, the method includes administering to a mammal an effective amount of a composition containing (i) albuterol, or a pharmaceutically acceptable derivative thereof; (ii) a buffer; and (iii) a metal chelator, whereby the disease or disorder is treated or prevented, or one or more symptoms are ameliorated. In one embodiment, the composition is an aqueous pharmaceutical composition for inhalation. The mammal treated is, in certain embodiments, a human.

In another embodiment, the method provided herein includes oral or nasal inhalation administration of a composition provided herein. In certain embodiments herein, the composition is administered to a patient in need of such treatment via nebulization.

The methods for treatment, prevention, or amelioration of one or more symptoms of bronchoconstrictive disorders, in another embodiment, further include administering one or more of (a) or (b) as follows: (a) an anti-inflammatory steroid; or (b) a dopamine (D₂) receptor agonist; simultaneously with, prior to or subsequent to the composition provided herein.

Anti-inflammatory steroids for use herein include, but are not limited to, beclomethasone dipropionate (BDP), beclomethasone monopropionate (BMP), flunisolide, triamcinolone acetonide, dexamethasone, tipredane, ciclesonid, rofleponide, mometasone, mometasone furoate (Asmanex® Twisthaler™, Shering-Plough Corporation, Kenilworth, N.J.), fluticasone or fluticasone propionate and budesonide, or derivatives thereof. In one embodiment, the steroidal anti-inflammatory is fluticasone, fluticasone propionate, budesonide, or a derivative thereof.

Dopamine (D₂) receptor agonists include, but are not limited to, Apomorphine ((R)-5,6,6a,7-tetrahydro-6-methyl-4H-dibenzo[de,g]quinoline-10,11-diol); Bromocriptine ((5′α)-2-bromo-12′-hydroxy-2′-(1-methylethyl)-5′-(2-methylpropyl)ergotaman-3′,6′,18-trione); Cabergoline ((8â)-N-(3-(dimethylamino)propyl)-N-((ethylamino)carbonyl)-6-(2-propenyl)ergoline-8-carboxamide); Lisuride (N′-((8a)-9,10-didehydro-6-methylergolin-8-yl)-N,N-diethylurea); Pergolide ((8α)-8-((methylthio)methyl)-6-propylergoline); Levodopa (3-hydroxy-L-tryrosine); Pramipexole ((S)-4,5,6,7-tetrahydro-N⁶-propyl-2,6-benzothiazolediamine); Quinpirole hydrochlrodie (trans-(−)-4aR-4,4a,5,6,7,8,8a,9-octahydro-5-propyl-1H-pyrazolo[3,4-g]quinoline hydrochloride); Ropinirole (4-(2-(dipropylamino)ethyl)-1,3-dihydro-2H-indol-2-one); and Talipexole (5,6,7,8-tetrahydro-6-(2-propenyl)-4H-thiazolo[4,5-d]azepin-2-amine). Other dopamine D₂ receptor agonists for use herein are disclosed in International Patent Application Publication No. WO 99/36095.

Other active ingredients for use herein in combination therapy, include, but are not limited to, IL-5 inhibitors such as those disclosed in U.S. Pat. Nos. 5,668,110, 5,683,983, 5,677,280 and 5,654,276; antisense modulators of IL-5 such as those disclosed in U.S. Pat. No. 6,136,603; milrinone (1,6-dihydro-2-methyl-6-oxo-[3,4′-bipyridine]-5-carbonitrile); milrinone lactate; tryptase inhibitors such as those disclosed in U.S. Pat. No. 5,525,623; tachykinin receptor antagonists such as those disclosed in U.S. Pat. Nos. 5,691,336, 5,877,191, 5,929,094, 5,750,549 and 5,780,467; leukotriene receptor antagonists such as montelukast sodium (Singular®, R-(E)]-1-[[[1-[3-[2-(7-chloro-2-quinolinyl)ethenyl]phenyl]-3-[2-(1-hydroxy-1-methylethyl)phenyl]propyl]thio]methyl]cyclopropaneacetic acid, monosodium salt), 5-lypoxygenase inhibitors such as zileuton (Zyflo®, Abbott Laboratories, Abbott Park, Ill.), and anti-IgE antibodies such as Xolair® (recombinant humanized anti-IgE monoclonal antibody (CGP 51901; IGE 025A; rhuMAb-E25), Genentech, Inc.).

The bronchoconstrictive disorder to be treated, prevented, or whose one or more symptoms are to be ameliorated is associated with asthma, including, but not limited to, bronchial asthma, allergic asthma and intrinsic asthma, e.g., late asthma and airway hyper-responsiveness; chronic bronchitis; and other chronic obstructive pulmonary diseases.

H. Nebulizers

In one embodiment, the compositions provided herein are intended for administration to a patient in need of such treatment via nebulization. Nebulizers that nebulize liquid formulations containing no propellant are suitable for use with the compositions provided herein. Nebulizers are available from, e.g., Pari GmbH (Starnberg, Germany), DeVilbiss Healthcare (Heston, Middlesex, UK), Healthdyne, Vital Signs, Baxter, Allied Health Care, Invacare, Hudson, Omron, Bremed, AirSep, Luminscope, Medisana, Mountain Medical, Aerosol Medical Ltd. (Colchester, Essex, UK), AFP Medical (Rugby, Warwickshire, UK), Bard Ltd. (Sunderland, UK), Carri-Med Ltd. (Dorking, UK), Plaem Nuiva (Brescia, Italy), Henleys Medical Supplies (London, UK), Intersurgical (Berkshire, UK), Lifecare Hospital Supplies (Leies, UK), Medic-Aid Ltd. (West Sussex, UK), Medix Ltd. (Essex, UK), Sinclair Medical Ltd. (Surrey, UK), and many others.

Nebulizers for use herein include, but are not limited to, jet nebulizers (optionally sold with compressors), ultrosonic nebulizers, and others. Exemplary jet nebulizers for use herein include Pari LC plus/ProNeb, Pari LC plus/ProNeb Turbo, Pari LC plus/Dura Neb 1000 & 2000, Pari LC plus/Walkhaler, Pari LC plus/Pari Master, Pari LC star, Plumo-aide, Pulmo-aide LT, Pulmo-aide traveler, Invacare Passport, Inspiration Healthdyne 626, Pulmo-Neb Traverler, DeVilbiss 646, Whisper Jet, Acorn II, Misty-Neb, Allied aerosol, Schuco Home Care, Lexan Plasic Pocet Neb, SideStream Hand Held Neb, Mobil Mist, Up-Draft, Up-Draft II, T Up-Draft, ISO-NEB, AVA-NEB, Micro Mist, and PulmoMate. Exemplary ultrasonic nebulizers for use herein include MicroAir, UltraAir, CompAir, Pulmosonic, Scout, 5003 Ultrasonic Neb, 5110 Ultrasonic Neb, 5004 Desk Ultrasonic Nebulizer, Mystique Ultrasonic, Luminscope's Ultrasonic Nebulizer, Medisana Ultrasonic Nebulizer, Microstat Ultrasonic Nebulizer, and MABISMist Hand Held Ultrasonic Nebulizer. Other nebulizers for use herein include 5000 Electromagnetic Neb, 5001 Electromagnetic Neb 5002 Rotary Piston Neb, Lumineb I Piston Nebulizer 5500, and AeroEclipse Breath Actuated Nebulizer.

I. Articles of Manufacture

The compositions provided herein may be packaged as articles of manufacture containing packaging material, a composition provided herein, which is useful for treatment, prevention or amelioration of one or more symptoms of diseases or disorders associated with undesired and/or uncontrolled bronchoconstriction, and a label that indicates that the composition is used for treatment, prevention or amelioration of one or more symptoms of diseases or disorders associated with undesired and/or uncontrolled bronchoconstriction.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. In one embodiment herein, the compositions are packaged with a nebulizer for administration of the composition to a patient in need thereof.

The following examples are provided for illustrative purposes only and are not intended to limit the scope of the appended claims.

### EXAMPLE 1

Preparation of Compositions

The compositions shown below in Table 1 were prepared in glass beakers using magnetic stir bars. The compositions were stored in amber colored bottles until use. The pH of all formulations was between 3.5 and 3.6 (target: 3.5).

TABLE 1

| | Albuterol | | | |
|---|---|---|---|---|
| | Sulfate (% | EDTA (% | Citric acid | 1N aq. H₂SO₄ (for pH |
| Description | w/w/) | w/w/) | % w/w/) | adjustment to 3.5) |
| Albuterol | 0.60 | — | — | q.s. |
| Alb 0.5% | | | | |
| Alb 0.5% + | 0.60 | — | 0.01 | — |
| Citric | | | | |
| acid | | | | |
| Alb 0.5% + | 0.60 | 0.01 | — | q.s. |
| EDTA | | | | |
| Alb 0.5% + | 0.60 | 0.01 | 0.01 | — |
| EDTA + | | | | |
| Citric | | | | |
| acid | | | | |

### EXAMPLE 2

Preparation of a Composition on Large Scale

A 100 kg batch or albuterol sulfate inhalation solution was manufactured and filled into plastic vials by form-fill-seal (FFS) technology. The batch was prepared with sodium citrate and hydrochloric acid so that citric acid was formed in situ at the desired concentration and at the same time the solution was buffered. The composition of the batch was as follows:

| | |
|---|---|
| Albuterol Sulfate | 600 g |
| EDTA disodium salt | 5 g |
| Sodium citrate, dihydrate | 100 g |
| Hydrochloric acid, 1N | 765 g |
| Water q.s. to | 100 kg |

### EXAMPLE 3

Analysis of Compositions

The compositions of Example 1 were transferred into scintillation vials (10 mL each). Vials were stored at 60° C. controlled conditions. All formulations were analyzed at time-zero for initial data. Samples were pulled after 1, 2, and 4 weeks and analyzed by HPLC (test method: column: YMC Phenyl column, 120 Å, 250×4.6 mm ID, 5 μm; mobile phase: 25 mM KH₂PO₄, pH 3.0/methanol (95/5, v/v); column temperature: ambient; flow rate: 1.5 mL/min; detection: UV at 225 nm; injection volume 20 μL; run time: 35 min; retention times:

| | | Relative Retention |
|---|---|---|
| Compound | Retention Time (min) | Time (RRT) |
| Albuterone | 7.3 | 0.88 |
| Albuterol | 8.1 | 1.0 |
| Chloroalbuterone | 11.9 | 1.5 |
| 5-Chloroalbuterol | 17.9 | 2.4 |
| Methyl albuterol | 20.1 | 2.7 |
| Albuterol aldehyde | 21.1 | 2.8 |
| Methoxymethyl albuterol | 26.1 | 3.5 |

Albuterone is tert-butylamino-4-hydroxy-5-hydroxymethylacetophenone; chloroalbuterone is tert-butylamino-3-chloro-4-hydroxy-5-hydroxymethylacetophenone; 5-chloroalbuterol is 1-(5-chloro-4-hydroxy-3-hydroxymethylphenyl)-2-(tert-butylamino)ethanol; methyl albuterol is 1-(4-hydroxy-3-methyphenyl)-2-(tert-butylamino)ethanol; albuterol aldehyde is 5-(2-((1,1-dimethylethyl)amino)-1-hydroxyethyl)-2-hydroxybenzaldehyde; and methoxymethyl albuterol is 2-tert-butylamino-1-(4-hydroxy-3-methoxymethylphenyl)-ethanol.

Table 2, below, summarizes the % w/w of albuterol aldehyde in test compositions (see, Example 1) after 1, 2 and 4 weeks of storage at 60° C. Significant differences in albuterol aldehyde levels were observed. Addition of either citric acid or EDTA decreased formation of albuterol aldehyde significantly from that of control. The level of albuterol aldehyde in test formulations pH adjusted with citric acid was 0.09% (as compared to 0.28% in formulations pH adjusted with sulfuric acid-control). Addition of EDTA 0.01% controlled the albuterol aldehyde level at 0.06%. Albuterol aldehyde level was contained further in test formulations containing EDTA and citric acid (0.05% as compared to 0.28% in control).

TABLE 2

| | 60° C. | | | |
|---|---|---|---|---|
| Description | Initial | 1 week | 2 weeks | 4 weeks |
| Alb 0.5% | ND | 0.04 | 0.11 | 0.28 |
| Alb 0.5% + Citric | ND | 0.006 | 0.04 | 0.09 |
| acid | | | | |
| Alb 0.5% + EDTA | ND | ND | 0.03 | 0.06 |
| Alb 0.5% + EDTA + Citric | ND | ND | 0.02 | 0.05 |
| acid | | | | |

### EXAMPLE 4

Analysis of Large Scale Composition

The composition of Example 2 was transferred into scintillation vials (10 mL each). Vials were stored at 60° C. controlled conditions. The formulation was analyzed at time-zero for initial data. Samples were pulled after 1, 2, 3 and months and analyzed by HPLC using the test method provided in Example 3.

Table 3, below, summarizes the area % of albuterol aldehyde in the composition (see, Example 2) after 1, 2, 3 and 6 months of storage at 40° C. and 75% relative humidity (RH). Also, this composition possessed only 0.02% (by area) of total degradation products after 6 months at 40° C./75% RH. Thus, the levels of albuterol aldehyde and total degradation products in this composition were reduced to 0.01% or lower, and 0.02% (by area) or lower, respectively.

TABLE 3

| Interval (months) | Albuterol Aldehyde (%) |
|---|---|
| 0 | ND |
| 1 | ND |
| 2 | 0.008 |
| 3 | 0.01 |
| 6 | ND |

Since modifications will be apparent to those of skill in the art, it is intended that the invention be limited only by the scope of the appended claims.

What is claimed is:

1. A pharmaceutical composition, comprising:
(i) albuterol, or a pharmaceutically acceptable derivative thereof;
(ii) a buffer; and
(iii) a metal chelator.

2. The pharmaceutical composition of claim 1, wherein the pharmaceutically acceptable derivative of albuterol is a pharmaceutically acceptable salt.

3. The pharmaceutical composition of claim 1, wherein the pharmaceutically acceptable derivative of albuterol is a mineral acid salt.

4. The pharmaceutical composition of claim 1, wherein the pharmaceutically acceptable derivative of albuterol is a sulfate salt.

5. The pharmaceutical composition of claim 1, wherein the pharmaceutically acceptable derivative of albuterol is albuterol hemisulfate, having the formula:

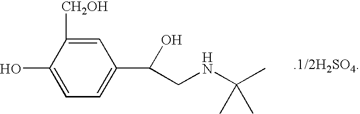

6. The pharmaceutical composition of claim 1, wherein the buffer is citric acid, citrate buffer, citric acid/phosphate buffer, phosphate-acetate-borate buffer (Britton-Robinson), and citrate-phosphate-borate buffer (Teorell-Stanhagen).

7. The pharmaceutical composition of claim 1, wherein the buffer is citric acid.

8. The pharmaceutical composition of claim 1, wherein the metal chelator is selected from ethylenediamine tetraacetic acid (EDTA, (HOOCCH₂)₂NCH₂CH₂N—(CH₂COOH)₂), nitrilotriacetic acid (N(CH₂COOH)₃), ethylene glycol-bis(β-aminoethyl ether)-N,N-tetraacetic acid (HOOCCH₂)₂NCH₂CH₂)CH₂CH₂O—CH₂CH₂N(CH₂COOH)₂), ethylene glycol bis(β-aminoethyl ether)-N,N,N′,N′-tetaacetic acid (EGTA), glycine, salicylaldehyde, albumin, pilocarpine, chlorophyll, hemoglobin, peroxidases, cytochromes, oxidases, ascorbic acid oxidase, tyrosinase, polyphenoloxidase, lactase, phosphatase, carboxylases, insulin, cyanocobalbumin, carbonic anhydrase, xanthine dehydrogenase and tetracyclines.

9. The pharmaceutical composition of claim 1, wherein the metal chelator is ethylenediaminetetraactic acid (EDTA) or a pharmaceutically acceptable salt thereof.

10. The pharmaceutical composition of claim 1, wherein the metal chelator is a salt of ethylenediaminetetraactic acid (EDTA).

11. The pharmaceutical composition of claim 1, wherein the metal chelator is a sodium salt of ethylenediaminetetraactic acid (EDTA).

12. The pharmaceutical composition of claim 1, wherein the metal chelator is ethylenediaminetetraactic acid (EDTA) disodium salt.

13. The pharmaceutical composition of claim 1 that is an aqueous composition.

14. A pharmaceutical composition, comprising albuterol, or a pharmaceutically acceptable derivative thereof, wherein the composition is an aqueous composition that is stable for at least 6 months at 40° C. or 18 months at 25° C.

15. A pharmaceutical composition, comprising albuterol, or a pharmaceutically acceptable derivative thereof, wherein the composition is not sparged with nitrogen and is stable for at least 6 months at 40° C. or 18 months at 25° C. after storage under an ambient atmosphere.

16. The pharmaceutical composition of claim 15, wherein less than 0.02 area % of total degradation products are present after 6 months at 40° C.

17. The pharmaceutical composition of claim 15, wherein less than 0.01% albuterol aldehyde is present after storage for 6 months at 40° C.

18. The pharmaceutical composition of claim 1, wherein the albuterol concentration is from about 0.01 weight % to about 0.75 weight %.

19. The pharmaceutical composition of claim 18, wherein the albuterol concentration is from about 0.01 weight % to about 0.5 weight %.

20. The pharmaceutical composition of claim 18, wherein the albuterol concentration is about 0.5 weight %.

21. The pharmaceutical composition of claim 12, wherein the EDTA disodium salt concentration is from about 0.005 weight % to about 0.25 weight %.

22. The pharmaceutical composition of claim 21, wherein the EDTA disodium salt concentration is about 0.01 weight %.

23. The pharmaceutical composition of claim 21, wherein the EDTA disodium salt concentration is about 0.005 weight %.

24. The pharmaceutical composition of claim 7, wherein the citric acid concentration is from about 0.005 weight % to about 0.25 weight %.

25. The pharmaceutical composition of claim 24, wherein the citric acid concentration is about 0.01 weight %.

26. The pharmaceutical composition of claim 1, wherein the pH of the composition is from about 3.0 up to about 5.0.

27. The pharmaceutical composition of claim 1, wherein the pH of the composition is about 3.5.

28. A combination, comprising:
(i) the pharmaceutical composition of claim 1; and
(ii) a vial.

29. The combination of claim 28, wherein the vial is a low density polyethylene (LDPE) or a polypropylene (PP) vial.

30. The combination of claim 28, wherein the vial contains 0.5 mL of the pharmaceutical composition.

31. The combination of claim 28, wherein the vial is a unit dose plastic molded vial.

32. The pharmaceutical composition of claim 1, further comprising an anti-inflammatory steroid; a dopamine (D₂) receptor agonist; an IL-5 inhibitors; an antisense modulators of IL-5; milrinone; milrinone lactate; a tryptase inhibitor; a tachykinin receptor antagonist; a leukotriene receptor antagonist; a 5-lypoxygenase inhibitor; and an anti-IgE antibody.

33. An article of manufacture, comprising packaging material, the pharmaceutical composition of claim 1, which is useful for treatment, prevention or amelioration of one or more symptoms of diseases or disorders associated with undesired and/or uncontrolled bronchoconstriction, and a label that indicates that the composition is used for treatment, prevention or amelioration of one or more symptoms of diseases or disorders associated with undesired and/or uncontrolled bronchoconstriction.

34. A method for treating, preventing, or ameliorating one or more symptoms of bronchoconstrictive disorders, comprising administering a composition of claim 1.

35. A kit, comprising:
(i) the pharmaceutical composition of claim 1; and
(ii) a nebulizer.

36. A method of stabilizing an albuterol inhalation pharmaceutical composition, comprising adding a buffer and a metal chelator to the composition.

37. A method of preparing an aqueous pharmaceutical composition of claim 1, comprising:
(i) dissolving the metal chelator in water;
(ii) adding buffer to the metal chelator solution;
(iii) adding albuterol to the buffer/metal chelator solution.

38. A method of reducing albuterol aldehyde in an albuterol inhalation pharmaceutical composition, comprising adding a buffer and a metal chelator to the composition.

39. The composition of claim 1, comprising albuterol hemisulfate at a concentration of about 0.6% by weight; EDTA disodium salt at a concentration of about 0.005% by weight; and sodium citrate dihydrate at a concentration of about 0.1% by weight.

40. The composition of claim 39, further comprising 1 N aqueous hydrochloric acid at a concentration of about 0.765% by weight.

41. The composition of claim 1, wherein the albuterol is albuterol hemisulfate; the buffer is sodium citrate dihydrate; and the metal chelator is EDTA disodium salt.

42. The method of claim 34, wherein the composition is administered by inhalation.

43. The method of claim 42, wherein the disorder is asthma, chronic bronchitis and chronic obstructive pulmonary disease.

44. The method of claim 43, wherein the asthma is bronchial asthma, allergic asthma, intrinsic asthma (e.g., late asthma) or airway hyper-responsiveness.

45. A combination selected from (i), (ii) or (iii) as follows:
(i) a combination, comprising (a) a composition comprising albuterol, or a pharmaceutically acceptable derivative thereof, and a buffer, and (b) a metal chelator;
(ii) a combination, comprising (a) a composition comprising albuterol, or a pharmaceutically acceptable derivative thereof, and a metal chelator; and (b) a buffer; or
(iii) a combination, comprising (a) a composition comprising a metal chelator and a buffer, and (b) albuterol, or a pharmaceutically acceptable derivative thereof.
